(19) United States
(12) Patent Application Publication
(10) Pub. No.: US 2024/0343689 A1
(43) Pub. Date: Oct. 17, 2024

(54) SYNTHESIS OF BRIVARACETAM

(71) Applicant: OPTIMUS DRUGS PRIVATE LIMITED, Hyderabad (IN)

(72) Inventors: Desireddy SRINIVASA REDDY, Hyderabad (IN); Peketi SUBBA REDDY, Hyderabad (IN); Vedururi MADHAVA REDDY, Hyderabad (IN)

(21) Appl. No.: 18/683,602

(22) PCT Filed: Aug. 6, 2022
(86) PCT No.: PCT/IB2022/057344

§ 371 Date: Feb. 14, 2024

(30) Foreign Application Priority Data
Aug. 16, 2021 (IN) 202141037050

(51) Int. Cl. C07D 207/27; C07D 307/30

(52) U.S. Cl. CPC C07D 207/27; C07D 307/30

(57) ABSTRACT

The present invention relates to a novel compound of(S)-2-((Z)-((R)-4-propyldihydrofuran-2(3H)-ylidene)amino) butanamide and an improved, commercially viable process for preparation of Brivaracetam using(S)-2-((Z)-((R)-4-propyldihydrofur an-2(3H)-ylidene)amino)butanamide.

### FIELD OF THE INVENTION

The present invention relates to a novel compound of (S)-2-((Z)-((R)-4-propyldihydrofuran-2(3H)-ylidene)amino)butanamide and an improved, commercially viable process for preparation of Brivaracetam using(S)-2-((Z)-((R)-4-propyldihydrofuran-2(3H)-ylidene)amino)butanamide.

### BACKGROUND OF THE INVENTION

Brivaracetam is an antiepileptic drug for the treatment of partial-onset seizures with or without secondary generalisation, in combination with other antiepileptic drugs. Brivaracetam is approved by USFDA in May 2016.

Brivaracetam is a racetam derivative with anticonvulsant properties and is 4-n-propyl analogue of Levetiracetam. Brivaracetam is chemically known as (2S)-2-[(4R)-2-oxo-4-propyl pyrrolidinyl] butanamide. Its empirical formula is C₁₁H₂₀N₂O₂ and the molecular weight is 212.29. The structural formula is:

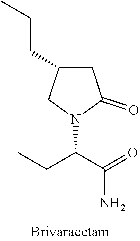

Brivaracetam is basically a chemical analogue of Levetiracetam, marketed under the brand name of BRIVIACT for the treatment as adjunctive therapy in the treatment of partial-onset seizures in patients at 16 years of age and older with epilepsy. Brivaracetam has an advantage over Levetiracetam in that it gets into the brain “much more quickly,” which means that “it could be used for status epilepticus, or acute seizures than cluster or prolonged seizures”. From the Phase III trials, the self-reported rate of irritability with Brivaracetam was 2% for both drug doses (100 mg and 200 mg) Vs 1% for placebo, which compares to as much as 10% for Levetiracetam in some post-marketing studies with the improved safety profile and possibility to be used for wider range of epilepsy, Brivaracetam is considered as one of the most promising 3rd generation antiepileptic drugs. Brivaracetam is reported in U.S. Pat. No. 6,784,197 by UCB, S.A. The synthetic process for Brivaracetam is reported in US '197, which comprises reacting 4-n-propyl-hydroxyfuranone with(S)-2-aminobutyramide in presence of toluene/H₂O/AcOH and NaBH₄ to obtain the compound of unsaturated pyrrolidone and its followed by treated with HCOONH₄/Pd/C/H₂O and preparative HPLC on chiral phase to obtain Brivaracetam.

The above process is schematically shown as below:

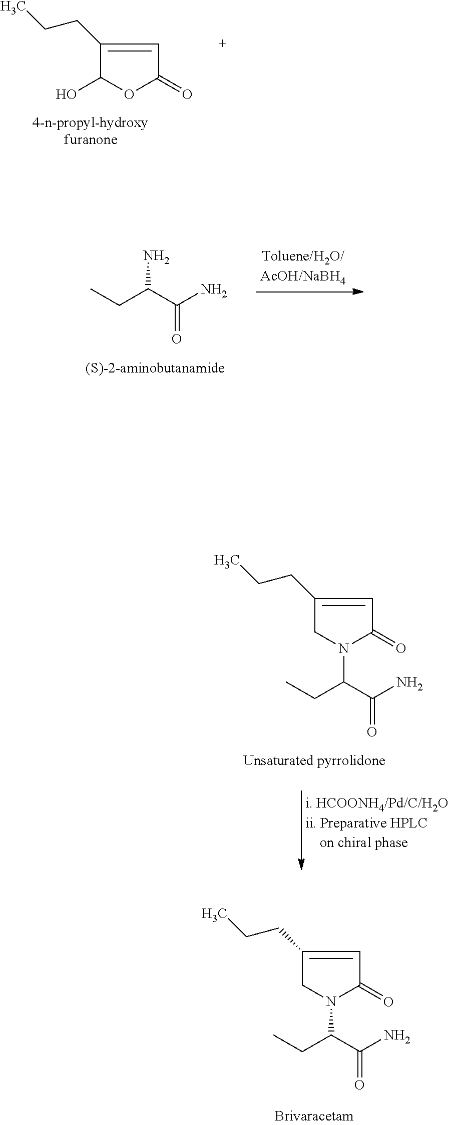

U.S. Pat. Nos. 762,947B2, 8,076,493B2 & 8,338,621B2 discloses the process for the preparation of Brivaracetam.

All the above processes disclose the preparation of the pure Brivaracetam involving the separation of enantiomers of the Brivaracetam using column chromatography, preparative HPLC it difficult for bulk manufacturing as well as it affects the overall yield making the process commercially.

Therefore, there is a need in the prior art for improved process for the preparation of Brivaracetam. In view of the foregoing, the present invention provides as result of extensive studies, process for the preparation of Brivaracetam using(S)-2-((Z)-((R)-4-propyldihydrofuran-2(3H)-ylidene)amino)butanamide. The advantage of the present invention w.r.t environmental variables, such as humidity, moisture content is eliminated from the manufacturing process. The present invention is providing a simple, cost effective with high purity and good yield on industrial applicable process.

### OBJECTIVE OF THE INVENTION

The objective of the present invention is to provide a method for the preparation of Brivaracetam by using novel compound of(S)-2-((Z)-((R)-4-propyldihydrofuran-2(3H)-ylidene)amino)butanamide.

In yet another objective of the present invention a novel compound of(S)-2-((Z)-((R)-4-propyldihydrofuran-2(3H)-ylidene)amino)butanamide.

### SUMMARY OF THE INVENTION

The present invention relates to a novel compound of(S)-2-((Z)-((R)-4-propyldihydrofuran-2(3H)-ylidene)amino) butanamide and an improved, commercially viable process for preparation of Brivaracetam using(S)-2-((Z)-((R)-4-propyldihydrofuran-2(3H)-ylidene)amino) butanamide.

In one aspect, the present invention provides a process for the preparation of Brivaracetam, comprising the steps of;
a) (R)-4-propyldihydro furan-2(3H)-one is reacted with(S)-2-aminobutanamide or HCl in presence or absence of base and organic solvent to obtain (R)-N-((S)-1-amino-1-oxobutan-2-yl)-3-(hydroxymethyl) hexanamide,

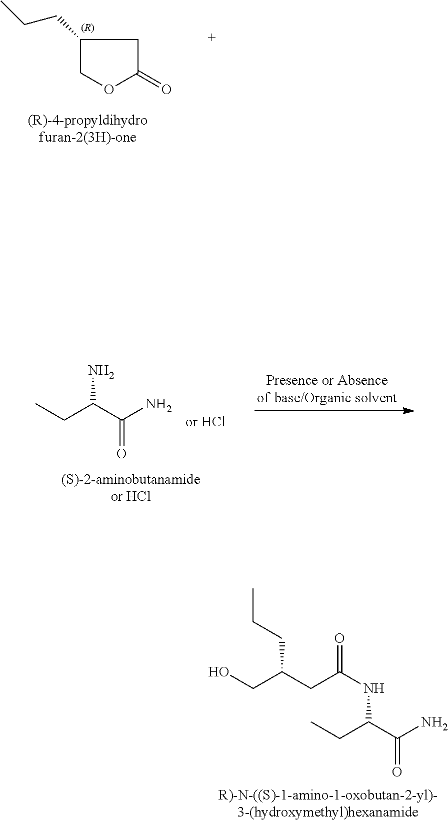

b) (R)-N-((S)-1-amino-1-oxobutan-2-yl)-3-(hydroxymethyl) hexanamide undergoes cyclisation in presence of acid to obtain(S)-2-((Z)-((R)-4-propyldihydrofuran-2(3H)-ylidene)amino) butanamide, and

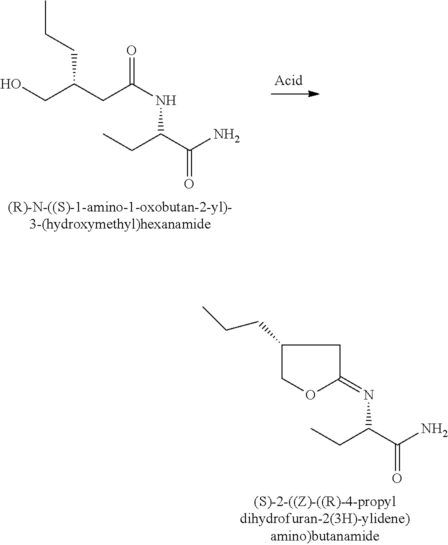

c) (S)-2-((Z)-((R)-4-propyldihydrofuran-2(3H)-ylidene)amino) butanamide converts into Brivaracetam in presence of acid/base and organic solvent.

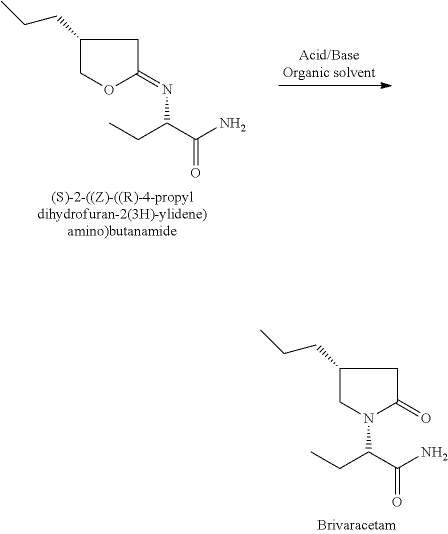

In yet another aspect, the present invention a novel compound of(S)-2-((Z)-((R)-4-propyldihydrofuran-2 (3H)-ylidene)amino) butanamide.

### DETAILED DESCRIPTION OF THE INVENTION

The present invention relates to a novel compound of(S)-2-((Z)-((R)-4-propyldihydrofuran-2(3H)-ylidene)amino) butanamide and an improved, commercially viable process for preparation of Brivaracetam using(S)-2-((Z)-((R)-4-propyldihydrofuran-2(3H)-ylidene)amino)butanamide.

In one aspect, the present invention provides a process for the preparation of Brivaracetam, comprising the steps of;
a) (R)-4-propyldihydro furan-2 (3H)-one is reacted with(S)-2-aminobutanamide or HCl in presence or absence of base and organic solvent to obtain (R)-N-((S)-1-amino-1-oxobutan-2-yl)-3-(hydroxymethyl) hexanamide,

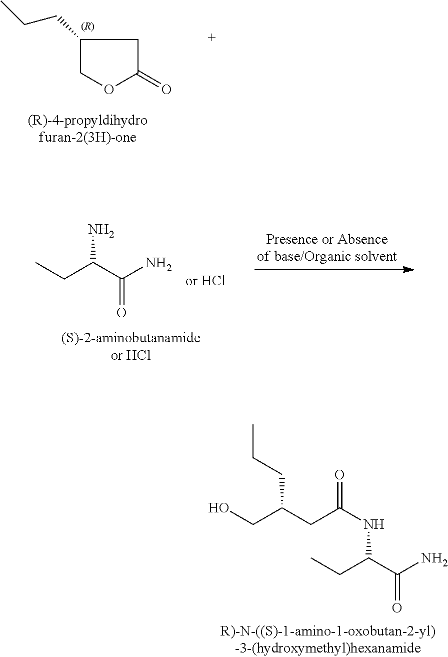

b) (R)-N-((S)-1-amino-1-oxobutan-2-yl)-3-(hydroxymethyl) hexanamide undergoes cyclisation in presence of acid to obtain(S)-2-((Z)-((R)-4-propyldihydrofuran-2(3H)-ylidene)amino) butanamide, and

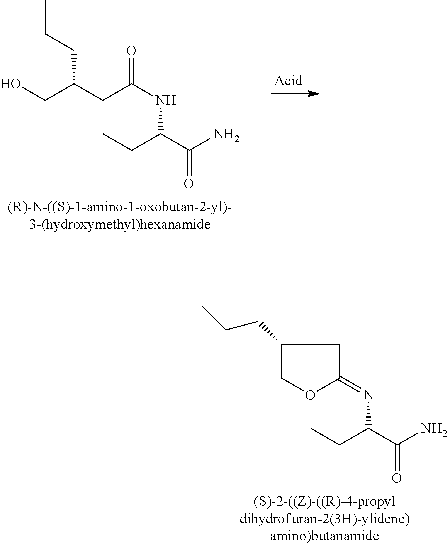

c) (S)-2-((Z)-((R)-4-propyldihydrofuran-2(3H)-ylidene)amino) butanamide converts into Brivaracetam in presence of acid/base and organic solvent.

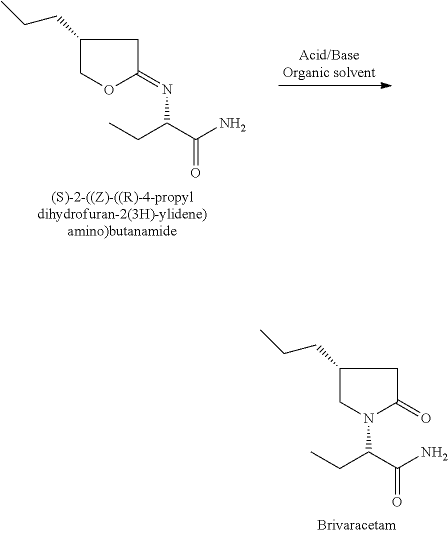

According to an embodiment of the present invention, Brivaracetam comprising by (R)-4-propyldihydro furan-2 (3H)-one is reacted with(S)-2-aminobutanamide or hydrochloric acid (HCl) in presence or absence of base and organic solvent to obtain (R)-N-((S)-1-amino-1-oxobutan-2-yl)-3-(hydroxymethyl) hexanamide and the reaction is carried out at reflux temperature for 2-24 hrs. (R)-N-((S)-1-amino-1-oxobutan-2-yl)-3-(hydroxymethyl) hexanamide undergoes cyclisation in presence of acid to obtain(S)-2-((Z)-((R)-4-propyldihydrofuran-2(3H)-ylidene) amino) butanamide and the reaction is carried out at 80-120° C. for 5-8 hrs. (S)-2-((Z)-((R)-4-propyldihydrofuran-2(3H)-ylidene)amino) butanamide converts into Brivaracetam in presence of acid and organic solvent and the reaction is carried out at 20-30° C. for 1-4 hrs, and then followed by the reaction in presence of base and organic solvent, optionally in presence of quaternary ammonium salt like tetra butyl ammonium bromide (TBAB) or dimethyl amino pyridine and the reaction is carried out at −30 to −10° C. for 4-8 hrs.

According to an embodiment of the present invention, Brivaracetam purifying by crude or tech solid of Brivaracetam with organic solvent and the reaction mixture was carried out at 40-45° C., followed by the reaction mixture was allow to cooled at 0-5° C. The obtained product washed with organic solvent, filtered to obtain pure compound of Brivaracetam.

According to an embodiment of the present invention, wherein the organic solvent is selected from alcohols such as methanol, ethanol, propanol, butanol, n-propyl alcohol, isopropyl alcohol, and t-butyl alcohol; nitriles such as acetonitrile and propionitrile; amides such as N,N-dimethylformamide and N,N-dimethylacetamide; sulfoxides such as dimethyl sulfoxide and diethyl sulfoxide; and aromatic hydrocarbons such as toluene, heptane and xylene; esters such as ethylacetate, methylacetate, butyl acetate, isopropyl acetate, methoxy ethyl acetate; ketones such as acetone, methylisobutyl ketone, 2-pentanone, ethylmethylketone, diethylketone; halogenated hydrocarbons such as chloroform, dichloromethane; ethers such as diethyl ether, tetrahydrofuran, dioxane or water and or mixtures thereof.

According to an embodiment of the present invention, wherein the base is selected from alkali metal hydroxides such as sodium hydroxide, lithium hydroxide or potassium hydroxide; alkali metal carbonates such as caesium carbonate, sodium carbonate potassium carbonate or lithium carbonate; alkali metal bicarbonates such as sodium bicarbonate or potassium bicarbonate; alkali metal alkoxides such as sodium methoxide, potassium methoxide, sodium ethoxide, potassium ethoxide, sodium tert-butoxide, potassium tert-butoxide, Lithium tert-butoxide; ammonia; and organic bases such as triethyl amine, methyl amine, ethyl amine, diisopropylethylamine; alkali halides such as sodium iodide, potassium iodide, lithium iodide and or mixtures.

According to an embodiment of the present invention, wherein the acid is selected from sulfuricacid, acetic acid, polyphosphoric acid, nitric acid, hydrochloric acid, hydrobromic acid, benzenesulfonyl chloride, ethanesulfonyl chloride and trifluoromethanesulfonic acid.

According to embodiment of the invention provides a process for preparation of pure Form-A or pure solid Phase 1 of Brivaracetam having purity of greater than 99.8%, comprising the steps of:
a. Crude Brivaracetam is dissolved in an organic solvent,
b. heating the reaction mixture at 30 to 50° C.,
c. cooling the reaction mixture at 0 to 10°° C.,
d. filtering the solid and wash with organic solvent,
e. isolating the pure Form-A or pure solid Phase 1 of Brivaracetam.

According to an embodiment of the present invention, wherein Brivaracetam is isolating as a pure Form A or pure solid Phase 1.

According to the embodiment of the present invention, the crystalline pure Form A or pure solid Phase 1 of Brivaracetam may have an XRPD pattern including diffraction peaks at 8.92, 10.0, 15.0, 15.74, 17.36, 19.19, 21.61, 24.99, 26.91, 32.59, 32.85, 37.81, 38.20, 39.32 and 43.97) (2θ±0.2°).

According to an embodiment of the present invention provides Brivaracetam having HPLC purity ≥99.8%.

In yet another aspect, the present invention a novel compound of(S)-2-((Z)-((R)-4-propyldihydrofuran-2 (3H)-ylidene)amino) butanamide.

The advantages of the present invention:
The present invention relates to a novel compound of(S)-2-((Z)-((R)-4-propyldihydrofuran-2 (3H)-ylidene)amino) butanamide.
The present invention an improved and commercially viable process for preparation of Brivaracetam using(S)-2-((Z)-((R)-4-propyldihydrofuran-2 (3H)-ylidene)amino) butanamide.
The present invention process for preparing Brivaracetam provide a simple, cost effective with high purity, good yield on eco-friendly and industrial applicable process.

The process details of the invention are provided in the examples given below, which are provided by way of illustration only and therefore should not be construed to limit the scope of the invention.

### EXAMPLES

### Example-1

Preparation of (R)-N-((S)-1-amino-1-oxobutan-2-yl)-3 (hydroxymethyl) hexanamide

Ethanol (800 ml), sodium hydroxide (37.5 gms) and(S)-2-aminobutanamide hydrochloride (120 gms) into round bottom flask (RBF) and stir for 1-2hrs. Filter the reaction mass, take filtrate into RBF, distilled out ethanol under vacuum at below 50° C. and charge (R)-4-propyldihydro furan-2 (3H)-one (100 gm) into RBF. The reaction mixture was heated at 95-100° C. and stir for 6-8 hrs at same temperature. After completion of the reaction, the reaction mass was allow to cooled at 65-70° C., further the reaction mass was allow to cooled at 25-30° C. and stir for 1-2hrs at same temperature, filter the material. The obtained product washed with ethyl acetate and suck dry the material. The obtained wet material into ethyl acetate (350 ml), the reaction mixture was heated at 45-50° C. and stir for 1-2 hrs.The obtained solid was filtered, dry the materials at hot air oven to get title compound.

Yield: 145gms (81.0%)

Purity: 96.0%

### Example-2

Preparation of (R)-N-((S)-1-amino-1-oxobutan-2-yl)-3-(hydroxymethyl) hexanamide

Ethanol (800 ml), sodium hydroxide (37.5 gms) and(S)-2-aminobutanamidehydrochloride (119 gms) into round bottom flask (RBF) and stir for 1-2 hrs. Filter the reaction mass, take filtrate into RBF, distilled out ethanol under vacuum at below 50°° C. and charge (R)-4-propyldihydro furan-2 (3H)-one (100 gm) into round bottom flask (RBF). The reaction mixture was heated at 95-100° C. and stir for 6-8 hrs at same temperature. After completion of the reaction, the reaction mass was allow to cooled to 65-70° C. and charge ethyl acetate (350 ml). The reaction mass was allow to cooled at 25-30° C. and stir for 1-2 hrs and filter the material. The obtained product washed with ethyl acetate and suck dry the material. The resultant wet material into ethyl acetate (350 ml), the reaction mixture was heated at 45-50° C. and stir for 1-2 hrs. The obtained solid was filtered, dry the materials at hot air oven to get title compound.

Yield: 150 gms (84.0%)

Purity: 96.0%

### Example-3

Preparation of (R)-N-((S)-1-amino-1-oxobutan-2-yl)-3-(hydroxymethyl) hexanamide

Charge ethanol (20 ml), (R)-4-propyldihydro furan-2 (3H)-one (5 gms) and(S)-2-amino butanamide (7.5gms) into round bottom flask (RBF) at 25-30° C. The reaction mas was heat to reflux temperature and maintained for 18-20 hrs at same temperature. After completion of the reaction, the reaction mas was cooled to room temperature. The obtained reaction mass was filtered through hyflo bed, distilled out ethanol completely and cool the reaction mass to add heptane into the reaction mass, stir for 1-2 hrs at room temperature. The obtained solid was filtered, dry the materials at hot air oven to get title compound.

Yield: 7.1gms (79.0%)

Purity: 96.0%

### Example-4

Preparation of (R)-N-((S)-1-amino-1-oxobutan-2-yl)-3-(hydroxymethyl) hexanamide

To a 100 ml three-necked flask with mechanical stirring function was added a 30% solution of sodium ethoxide in ethanol (9.10 g, 40.0 mmol), followed by added (S)-2-aminobutyramide hydrochloride (5.50 g, 40.0 mmol), stir for 0.5 hrs at room temperature. (4R)-4-propyl-dihydrofuran-2 (3H)-one (5.65 g, 20.0 mmol) was added to the obtained reaction mixture. The resulting mixture was heated to reflux temperature and stirred for 24 hrs, then water (50 ml) was added and the mixture was stirred for 1 hr. The resultant product was allowing to cool at room temperature, the solid was collected by filtration and washed with water to obtained crude product (6.67 g). The crude product was slurried with heptane to get pure title compound.

Yield: 8.5 g (84.0%).

Purity: 96.0%

### Example-5

Preparation of (R)-N-((S)-1-amino-1-oxobutan-2-yl)-3-(hydroxymethyl) hexanamide

To a 100 ml three-necked flask with mechanical stirring function was added a 30% solution of sodium hydroxide in ethanol (9.15 g, 40.0 mmol), followed by added(S) -2-aminobutyramide hydrochloride (5.55 g, 41.0 mmol), stir for 0.5 hrs at room temperature. (4R)-4-propyl-dihydrofuran-2 (3H)-one (5.70 g, 21.0 mmol) was added to the obtained reaction mixture. The resulting mixture was heated to reflux temperature and stirred for 24 hrs, then water (50 ml) was added and the mixture was stirred for 1 hr. The resultant product was allowing to cool at room temperature, the solid was collected by filtration and washed with water to obtained crude product (6.72 g). The crude product was slurried with heptane to get pure title compound.

Yield: 8.5g (83.0%).

Purity: 96.0%

### Example-6

Preparation of (R)-N-((S)-1-amino-1-oxobutan-2-yl)-3-(hydroxymethyl) hexanamide

To a 100 ml three-necked flask with mechanical stirring function was added(S)-2-aminobutyramide (4.10 g, 40.0 mmol) and MeOH (4 ml), followed by added (4R)-4-propyl-dihydrofuran-2(3H)-one (5.0 g). The resulting mixture was heated to reflux temperature and stirred for 24 hrs. Then 10% brine (50 ml) was added and the mixture was stirred for 1 hr. After completion of the reaction allowed to room temperature, the solid was collected by filtration, and washed with water, dried to obtain crude compound (6.08 g). The obtained crude product was recrystallized with heptane to get pure title compound.

Yield: 7.3 g (81.0%).

Purity: 96.0%

### Example-7

Preparation of (S)-2-((Z)-((R)-4-propyldihydrofuran-2(3H)-ylidene) amino) butanamide

To a 250 ml three-necked flask with mechanical stirrer was added (R)-N-((S)-1-amino-1-oxobutan-2-yl)-3-(hydroxymethyl) hexanamide (100 gms), charge conc. sulphuric acid (200 ml) at room temperature. The reaction mixture was heated to 95-100° C. and stirred for 6-8 hrs. After completion of the reaction, the reaction mass was cooled to room temperature, quenched in mixture of purified water (1600 ml), NaOH (320 gms) at below 20° C. and stirred for 1 hr, then added ethyl acetate (400 ml). The organic phase was collected after standing a while, washed with saturated brine (40 ml), dried with anhydrous Na₂SO₄ and concentrated. diisopropylether (200 ml) charged into mass and stirred for 1-2 hrs at 25-30° C., filter the materials, suck dry the materials to get title compound.

Yield: 75 g (82.0%).

Purity: 98.6%.

IR (cm−1): 3414.34, 2962, 1707, 1368.

1H-NMR (400 MHZ, CDCl3): 0.872-0.956 (m,6H), 1.296-1.457 (m,4H), 1.640-1.733 (m,2H), 1.733-1.920 (m,1H), 2.245-2.305 (m,1H), 2.385-2.459 (m,1H), 3.837-3.877 (t,1H), 4.146-4.176 (t,1H) 4.312-4.357 (t,1H), 6.824 (s, NH) 5.426 (s, NH)

### Example-8

Preparation of (S)-2-((Z)-((R)-4-propyldihydrofuran-2(3H)-ylidene) amino) butanamide

To a 250 ml three-necked flask with mechanical stirrer was added (R)-N-((S)-1-amino-1-oxobutan-2-yl)-3-(hydroxymethyl) hexanamide (100 gms), charge conc. sulphuric acid (200 ml) at room temperature. The reaction mixture was heated to 45-50° C. and stirred for 6-8 hrs. After completion of the reaction, the reaction mass was cooled to room temperature, quenched in mixture of purified water (1600 ml), NaOH (320 gms) at below 20° C. and stirred for 1 hr, then added ethyl acetate (400 ml). The organic phase was collected after standing a while, washed with saturated brine (40 ml), dried with anhydrous Na₂SO₄ and concentrated. diisopropylether (200 ml) charged into mass and stirred for 1-2 hrs at 25-30° C., filter the materials, suck dry the materials to get title compound.

Yield: 76 g (83.0%).

Purity: 98.6%

### Example-9

Preparation of (S)-2-((Z)-((R)-4-propyldihydrofuran-2(3H)-ylidene) amino) butanamide

To a 250 ml three-necked flask with mechanical stirring function was added (R)-N-((S)-1-amino-1-oxobutan-2-yl)-3-(hydroxymethyl) hexanamide (5.00 g,13.0 mmol), sulphuric acid (10 ml) at room temperature. The resulting reaction mixture was heated to 95-100° C. and stirred for 6-8 hrs. After completion of the reaction, the reaction mas was cooled to room temperature, add water (35 ml), NaOH (520 mg) and the reaction mixture was stirred for 1 hr, then added ethylacetate (20 ml). The organic phase was collected after standing a while, washed with saturated brine (40 ml), dried with anhydrous Na₂SO₄ and concentrated to get title product.

Yield: 3.8 g (82.0%).

Purity: 98.5%

### Example-10

Preparation of(S)-2-((R)-2-oxo-4-propylpyrrolidin-1-yl) butanamide (Brivaracetam)

To a 3000 ml three-necked flask with mechanical stirring function was added (S)-2-((Z)-((R)-4-propyldihydrofuran-2(3H)-ylidene)amino)butanamide (100 g), dichloromethane (1000 ml), purge HCl gas into the reaction mass at 25-30° C. and stir for 2-3 hrs, distilled out dichloromethane. The reaction mass was allow to cooled at −20 to −15 and charge dichloromethane (1000 ml), TBAB (11.4 gm), KOH powder (105.7 gms) and stir for 5-6 hrs at same temperature. After completion of the reaction, charge 5% Aqueous sodium bicarbonate solution into reaction mass and stir for 10-15 min. The reaction mixture was separate the two layers and extract product with dichloromethane. The obtained product dried with anhydrous Na₂SO₄, distilled out. The obtain product was added isopropyl acetate (100 ml), cool to 0-5° C. and stir for 2 hours, filtered. The obtain product was washed with n-heptane (15ml) to get crude title compound

Yield: 85 g (85.0%)

Purity: 99.2%

### Example-11

Preparation of(S)-2-((R)-2-oxo-4-propylpyrrolidin-1-yl) butanamide (Brivaracetam)

To a 3000 ml three-necked flask with mechanical stirring function was added (S)-2-((Z)-((R)-4-propyldihydrofuran-2(3H)-ylidene)amino)butanamide (100 g), dichloromethane (1000 ml), DMSO (5 ml) and purge HCl gas into the reaction mass at 25-30° C. and stir for 2-3hrs, distilled out dichloromethane. The reaction mass was allow to cooled at −20 to −15 and charge dichloromethane (1000 ml), TBAB (11.4 gm,), KOH powder (105.7 gms) and stir for 5-6 hrs at same temperature. After completion of the reaction, charge 5% Aqueous sodium bicarbonate solution into reaction mass and stir for 10-15 min. The reaction mixture was separate the two layers and extract product with dichloromethane. The obtained product dried with anhydrous Na₂SO₄, distilled out. The obtain product was added isopropyl acetate (100 ml), cool to 0-5° C. and stir for 2 hours, filtered. The obtain product was washed with n-heptane (10 ml) to get crude title compound

Yield: 83 g (83.0%)

Purity: 99.2%

### Example-12

Preparation of (S)-2-((R)-2-oxo-4-propylpyrrolidin-1-yl) butanamide (Brivaracetam)

To a 3000 ml three-necked flask with mechanical stirring function was added (S)-2-((Z)-((R)-4-propyldihydrofuran-2(3H)-ylidene)amino) butanamide (100 g), dichloromethane (1000 ml), purge HCl gas into the reaction mass at 25-30° C. and stir for 2-3hrs, distilled out dichloromethane. The reaction mass was allow to cooled at −15 to −10 and charge dichloromethane (1000 ml), TBAB (11.4 gm), KOH powder (105.7 gms) and stir 5-6 hrs at same temperature. After completion of the reaction, charge 5% aqueous sodium bicarbonate solution into reaction mass and stir for 10-15 min. The reaction mixture was separate the two layers and extract product with dichloromethane. The obtained product dried with anhydrous Na₂SO₄, distilled out. The obtain product was added isopropyl acetate (200 ml), cool to 0-5° C. and stir for 2 hours, filtered. The obtain product was washed with n-heptane (45 ml) to get crude title compound.

Yield: 86 g (86.0%)

Purity: 99.4%

### Example-13

Preparation of(S)-2-((R)-2-oxo-4-propylpyrrolidin-1-yl) butanamide (Brivaracetam)

To a 3000 ml three-necked flask with mechanical stirring function was added (S)-2-((Z)-((R)-4-propyldihydrofuran-2(3H)-ylidene)amino) butanamide (100 g), dichloromethane (1000 ml), DMSO (5 ml) and purge HCl gas into the reaction mass at 25-30° C. and stir for 2-3 hrs, distilled out dichloromethane. The reaction mass was allow to cooled at −15 to −10 and charge dichloromethane (1000 ml), TBAB (11.4 gm), KOH powder (105.7 gms) and stir 5-6 hrs at same temperature. After completion of the reaction, charge 5% aqueous sodium bicarbonate solution into reaction mass and stir for 10-15 min. The reaction mixture was separate the two layers and extract product with dichloromethane. The obtained product dried with anhydrous Na₂SO₄, distilled out. The obtain product was added isopropyl acetate (200 ml), cool to 0-5° C. and stir for 2 hours, filtered. The obtain product was washed with n-heptane (45 ml) to get crude title compound

Yield: 85 g (85.0%)

Purity: 99.4%

### Example-14

Preparation of(S)-2-((R)-2-oxo-4-propylpyrrolidin-1-yl)butanamide (Brivaracetam)

To a 250 ml three-necked flask with mechanical stirring function was added(S)-2-((Z)-((R)-4-propyldihydrofuran-2(3H)-ylidene)amino) butanamide (5.00 g), dichloromethane (50 ml) and pass HCl gas over 2-3 hrs at room temperature, added brine solution, stir for 30 min. The organic phase was collected after standing a while, washed with saturated brine (40 ml), dried with anhydrous Na₂SO₄, and concentrated. The resulting crude was charged with tetrahydrofuran (150 ml) under nitrogen atmosphere and cooled to −30 to −20° C., charged lot wise potassium tertbutoxide (2.3 gms) and stirred for 4-5 hrs. After completion of the reaction, the reaction mas was cooled to room temperature, followed by added saturated ammonium chloride solution (35 ml), ethylacetate (50 ml) and the mixture was stirred for 30 min. The organic phase was collected after standing a while, dried with anhydrous Na₂SO₄, and concentrated, isopropylacetate (10 ml) was added, cool to 0-5° C. to get title product.

Yield-3.9 g (78.0%).

Purity: 99.2.

### Example-15

Preparation of(S)-2-((R)-2-oxo-4-propylpyrrolidin-1-yl)butanamide (Brivaracetam)

To a 250 ml three-necked flask with mechanical stirring function was added(S)-2-((Z)-((R)-4-propyldihydrofuran-2(3H)-ylidene)amino) butanamide (5.00 g), dichloromethane (50 ml) and triethylamine (5 ml). The resultant mixture was cooled to 0-5° C., followed by slowly added benzene sulphonyl chloride and stir for 9-10 hrs at room temperature, added brine solution and stir for 30 min. The organic phase was collected after standing a while, washed with saturated brine (40 ml), dried with anhydrous Na₂SO₄ and concentrated. The resulting crude was charged with tetrahydrofuran (150 ml) under nitrogen atmosphere and the reaction mass was cooled to-20 to 30° C., charged lot wise potassium tertbutoxide (2.3 gms) and stirred for 4-5 hrs. After completion of the reaction, the reaction mass was cooled to room temperature, followed by added saturated ammonium chloride solution (35 ml), ethylacetate (50 ml) and the mixture was stirred for 30 min. The organic phase was collected after standing a while, dried with anhydrous Na₂SO₄, and concentrated, isopropyl acetate (10 ml) was added, cool to 0-5° C. to get title compound.

Yield: 4g (80.0%).

Purity: 99.2%

### Example-16

Preparation of(S)-2-((R)-2-oxo-4-propylpyrrolidin-1-yl)butanamide (Brivaracetam)

To a 250 ml three-necked flask with mechanical stirring function was added(S)-2-((Z)-((R)-4-propyldihydrofuran-2(3H)-ylidene)amino) butanamide (10.00 g), dichloromethane (100 ml) and triethylamine (5 ml). The resultant mixture was cooled to 0-5° C., followed by slowly added benzene sulphonyl chloride (9.5 g) and stir for 9-10 hrs at room temperature, added brine solution and stir for 30 min. The organic phase was collected after standing a while, washed with saturated brine (80 ml), dried with anhydrous Na₂SO₄ and concentrated. The resulting crude was charged with DMF (100 ml) under nitrogen atmosphere, followed by added potassium carbonate (15 gm), potassium iodide (4 gm) at room temperature and stirred for 20 hrs at 75-80°° C. After completion of the reaction, the reaction mass was cooled to room temperature and added brine solution (35 ml), ethylacetate (100 ml) and the reaction mixture was stirred for 30 min. The organic phase was collected after standing a while, dried with anhydrous Na₂SO₄ and concentrated, isopropyl acetate (10 ml) was added, coot to 0-5° C. to get titled compound. Yield: 7.9 g (79.0%).

Purity: 99.2%

### Example-17

Preparation of(S)-2-((R)-2-oxo-4-propylpyrrolidin-1-yl) butanamide (Brivaracetam)

To a 250 ml three-necked flask with mechanical stirring function was added(S)-2-((Z)-((R)-4-propyldihydrofuran-2(3H)-ylidene)amino) butanamide (10 g), dichloromethane (100 ml) and triethylamine (5 ml). The reaction mixture was cooled to 0-5° C., followed by slowly added ethane sulphonyl chloride (10.5 g) and stir for 9-10 hrs at room temperature, added brine solution and stir for 30 min. The organic phase was collected after standing a while, washed with saturated brine (80 ml), dried with anhydrous Na₂SO₄, and concentrated. The resulting crude was charged with DMF (100 ml) under nitrogen atmosphere, followed by added potassium carbonate (15 gm), potassium iodide (4 gm) at room temperature and stirred for 20 hrs at 75-80° C. After completion of the reaction, the reaction mass was cooled to room temperature, followed by added brine solution (35 ml), ethylacetate (100 ml) and the mixture was stirred for 30 min. The organic phase was collected after standing a while, dried with anhydrous Na₂SO₄, and concentrated, isopropyl acetate (10 ml) was added, cool to 0-5° C. to get title compound.

Yield: 7.8 g (78.0%).

Purity: 99.2%

### Example-18

Purification process of Brivaracetam

Take crude or tech solid of Brivaracetam (100 gr) and isopropyl acetate (200 ml) into round-bottom flask. The reaction mixture was heated at 40-45° C. and stir for 30 minutes. The reaction mixture was allow to cooled at 0-5° C. and stir for 1-2 hours. The product was filtered and washed with n-heptane (10 ml), filtered. The obtain solid dry under hot air oven to get pure title compound of Brivaracetam.

Yield: 94 g (94.0%)

Purity: 99.9%

1. A process for the preparation of Brivaracetam, comprising the steps of:
a) reacting (R)-4-propyldihydro furan-2(3H)-one is with (S)-2-aminobutanamide or HCl, optionally in the presence of base and/or organic solvent, to obtain (R)-N-((S)-1-amino-1-oxobutan-2-yl)-3-(hydroxymethyl) hexanamide,
b) cyclizing (R)-N-((S)-1-amino-1-oxobutan-2-yl)-3-(hydroxymethyl) hexanamide in presence of acid to obtain(S)-2-((Z)-((R)-4-propyldihydrofuran-2 (3H)-ylidene)amino) butanamide, and
c) converting(S)-2-((Z)-((R)-4-propyldihydrofuran-2(3H)-ylidene)amino) butanamide into Brivaracetam, optionally in presence of acid, base, and/or organic solvent.

2. The process of claim 1, wherein the organic solvent is selected from the group consisting of alcohols, nitriles; amides, sulfoxides, aromatic hydrocarbons, esters, ketones, halogenated hydrocarbons, ethers, and mixtures thereof.

3. The process of claim 1, wherein the base is selected from the group consisting of alkali metal hydroxides, alkali metal carbonates, alkali metal bicarbonates, alkali metal alkoxides, ammonia, organic bases alkali halides, and mixtures thereof.

4. The process of claim 1, wherein the acid is selected from the group consisting of sulfuric acid, acetic acid, polyphosphoric acid, nitric acid, hydrochloric acid, hydrobromic acid, benzenesulfonyl chloride, ethanesulfonyl chloride, and trifluoromethanesulfonic acid.

5. A compound of(S)-2-((Z)-((R)-4-propyldihydrofuran-2(3H)-ylidene)amino) butanamide.

6. (canceled)

7. The process of claim 2 wherein the organic solvent is an alcohol selected from the group consisting of methanol, ethanol, propanol, butanol, n-propyl alcohol, isopropyl alcohol, and t-butyl alcohol.

8. The process of claim 2 wherein the organic solvent is a nitrile selected from the group consisting of acetonitrile and propionitrile.

9. The process of claim 2 wherein the organic solvent is an amide selected from the group consisting of N,N-dimethylformamide and N,N-dimethylacetamide.

10. The process of claim 2 wherein the organic solvent is a sulfoxide selected from the group consisting of dimethyl sulfoxide and diethyl sulfoxide.

11. The process of claim 2 wherein the organic solvent is an aromatic hydrocarbon selected from the group consisting of toluene, heptane, and xylene.

12. The process of claim 2 wherein the organic solvent is an ester selected from the group consisting of ethylacetate, methylacetate, butyl acetate, isopropyl acetate, and methoxy ethyl acetate.

13. The process of claim 2 wherein the organic solvent is a ketone selected from the group consisting of acetone, methylisobutyl ketone, 2-pentanone, ethylmethylketone, and diethylketone.

14. The process of claim 2 wherein the organic solvent is a halogenated hydrocarbon selected from the group consisting of chloroform and dichloromethane.

15. The process of claim 2 wherein the organic solvent is an ether selected from the group consisting of diethyl ether, tetrahydrofuran, and dioxane.

16. The process of claim 3 wherein the base is an alkali metal hydroxide selected from the group consisting of sodium hydroxide, lithium hydroxide, and potassium hydroxide.

17. The process of claim 3 wherein the base is an alkali metal carbonate or alkali metal bicarbonate selected from the group consisting of caesium carbonate, sodium carbonate, potassium carbonate, lithium carbonate, sodium bicarbonate and potassium bicarbonate.

18. The process of claim 3 wherein the base is an alkali metal alkoxide selected from the group consisting of sodium methoxide, potassium methoxide, sodium ethoxide, potassium ethoxide, sodium tert-butoxide, potassium tert-butoxide, and lithium tert-butoxide.

19. The process of claim 3 wherein the base is ammonia.

20. The process of claim 3 wherein the base is an organic base selected from the group consisting of triethyl amine, methyl amine, ethyl amine, and diisopropylethylamine.

21. The process of claim 3 wherein the base is an alkali halide selected from the group consisting of sodium iodide, potassium iodide, and lithium iodide.
